SEQUENCE LISTING (6 sequences)

SEQ ID NO: 1 - DNA, 20 bp, artificial sequence
  synthetic oligonucleotide
aggcctcagtttccttgctc

SEQ ID NO: 2 - DNA, 21 bp, artificial sequence
  synthetic oligonucleotide
tgctgtctgttgtttcaccct

SEQ ID NO: 3 - DNA, 146 bp, homo sapiens
aggcctcagtttccttgctcacagatgtggatcccaggagctgcctcaaatacttggaggcatgaatggttctaaatcacgcatgccatgcctgacacattaaccccccattctcttcactgtgcagggtgaaacaacagacagca

SEQ ID NO: 4 - DNA, 313 bp, homo sapiens
tgggcaaatggggtatacatttcccctttaccagtgcatctgggcaaatggggtatacatctcctttttccccgtctggggagagaacaatagctttgtgagattcatggcccccaaagtccagcatcttccggtacccctcctccctccttagcccatttgcttcctatgatggctgcaaagagagaaacttgctgttggaaacagggggccttgggagtcaggctaagggattggaccttcctcctcacccaatgaaactgagagaagaacctgcattttccctggggtgggatgcactggtaaaggggaa

SEQ ID NO: 5 - DNA, 186 bp, homo sapiens
aggcctcagtttccttgctcgctgtctgttgtttcaccctaggcctcagtttccttgctcacagatgtggatcccaggagctgcctcaaatacttggaggcatgaatggttctaaatcacgcatgccatgcctgacacattaaccccccattctcttcactgtgcagggtgaaacaacagacagca

SEQ ID NO: 6 - DNA, 170 bp, homo sapiens
ggcatcagcctattgcatttctgttgcctgcaattcctttggcatcagcctattgcatttacttcttttgcctaaaatgtgatccttggttagaagccatgtatcattatgatgaataaggattttgttaagtccatggatggtattgctaaaggaattgcaggcaacag

(19) United States
(12) Patent Application Publication
(10) Pub. No.: US 2019/0360040 A1
(43) Pub. Date: Nov. 28, 2019

(54) COMPOSITIONS AND METHODS FOR PREDICTING ANALYTIC SUCCESS OF T-CELL QUANTIFICATION AND T-CELL RECEPTOR SEQUENCING

(71) Applicant: UNIVERSITY OF SOUTHERN CALIFORNIA

(72) Inventors: Stephen B. GRUBER, Los Angeles, CA (US); Marilena MELAS, Los Angeles, CA (US); Kevin MCDONNELL, Los Angeles, CA (US)

(21) Appl. No.: 16/487,045

(22) PCT Filed: Feb. 20, 2018
(86) PCT No.: PCT/US18/18681

§ 371 Date: Aug. 19, 2019

Related U.S. Application Data
(60) Provisional application No. 62/460,983, filed on Feb. 20, 2017.

(51) Int. Cl. C12Q 1/6869; C12Q 1/6881; G16H 50/30

(52) U.S. Cl. CPC C12Q 1/6869; C12Q 2535/122; G16H 50/30; C12Q 1/6881

(57) ABSTRACT

Provided are compositions, including products of manufacture and kits, and methods, for predicting the successful construction of a genomic T-Cell Receptor (TCR) library from a tissue sample; or predicting the successful sequencing of a TCR sequence from a tissue sample, wherein optionally the tissue sample comprises or is derived from a biopsy or a Formalin-Fixation and Paraffin Embedding (FFPE) sample, and optionally the tissue sample is a human or an animal sample. In alternative embodiments, provided herein are assays for predicting successful library construction and sequencing of a TCR chain. In alternative embodiments, the T cell is a tumor infiltrating lymphocyte (TIL). The results of methods provided herein can generate a prediction as to the success of assays used for quantifying T-cells and sequencing T-cell receptors, including any commercially available assay (e.g., ImmunoSeq, Adaptive Biotechnologies, Seattle, Wash.) to effectively assess TCR quantity and clonality.

### RELATED APPLICATIONS

This Patent Convention Treaty (PCT) International Application claims the benefit of priority to U.S. Provisional Application No. 62/460,983, filed Feb. 20, 2017. The aforementioned application is expressly incorporated herein by reference in its entirety and for all purposes.

### TECHNICAL FIELD

This invention generally relates to genomic sequencing and cancer diagnosis. In alternative embodiments, provided are compositions, including products of manufacture and kits, and methods, for predicting the successful construction of a genomic T-Cell Receptor (TCR) library from a tissue sample; or predicting the successful sequencing of a TCR sequence from a tissue sample, wherein optionally the tissue sample comprises or is derived from a biopsy or a Formalin-Fixation and Paraffin Embedding (FFPE) sample, and optionally the tissue sample is a human or an animal sample. In alternative embodiments, provided herein are assays for predicting successful library construction and sequencing of a TCR chain. In alternative embodiments, the T cell is a tumor infiltrating lymphocyte (TIL). The results of methods provided herein can generate a prediction as to the success of assays used for quantifying T-cells and sequencing T-cell receptors, including any commercially available assay (e.g., ImmunoSeq, Adaptive Biotechnologies, Seattle, Wash.) to effectively assess TCR quantity and clonality.

### BACKGROUND

The quantification and determination of the clonality of tumor infiltrating lymphocytes (TILs) reliably measures the patient's “immunocompetence” and may predict the response to immune-modulating cancer therapies. Assessment of TIL quantity and clonality can be achieved by next generation sequencing of T-Cell receptor (TCR) DNA (genomic) sequences (e.g. ImmunoSEQ™ Assay by Adaptive Biotechnologies, Seattle, Wash.) which uses optimized PCR primers and a synthetic immune repertoire with computational algorithms to sequence T cell receptor (TCR) genomic sequences, see e.g., U.S. Pat. Nos. 9,150,905 and 9,371,558). Preliminary data in a variety of tumor types including colorectal cancer, ovarian cancer and glioblastoma suggest that higher levels of clonally expanded TILs and percent tumor infiltration correlate with better patient outcomes and these metrics may serve as predictive biomarkers of response.

The TCR receptor family comprises 4 receptor chains: the TCR alpha, beta, gamma and delta chains. The loci for the alpha, beta and gamma chains encoding these TCRs are located respectively at the chromosomal positions 14q11-12, 7q32-35, 7p15. The delta chain gene locus is co-situated within the alpha gene locus at 14q11-12. Next-generation sequencing of the TCR loci requires preliminary construction of a sequencing library which itself depends on the ability to PCR amplify the TCR gene sequence. The size of the PCR amplicon required for construction of informative sequencing libraries ranges between 50 and 200 base pairs.

Formalin-Fixation and Paraffin Embedding (FFPE) is the most common procedure for preserving tissue for pathologic assessment, and FFPE preserved tissue represents the primary source of DNA for evaluation of tumor TIL quantity and clonality. Because of degradation effects associated with the formalin fixation process and aging, the DNA from FFPE samples is often fragmented; as well, as tissue specimens are frequently of modest amounts (e.g., needle biopsy specimens) the quantity of DNA available from these specimens is often quite limited. These qualitative and quantitative limitations of the DNA derived from FFPE samples may compromise the ability to construct acceptable DNA libraries required to perform genomic sequencing, e.g., next generation sequencing, and therefore TIL assessment dependent on that sequencing.

The construction and sequencing of a next generation sequencing DNA library is expensive and time consuming costing many hundreds of dollars and requiring numerous man hours of labor. Failed library construction represents a significant research opportunity cost as the resources devoted to the failed library construction might have otherwise be employed towards construction of a successful, informative library. Therefore, an ability to predict whether FFPE DNA quality and quantity are sufficient to result in successful library construction and next generation sequencing of the TCR loci represents a tremendous cost saving measure and facilitates research efficiency.

### SUMMARY

In alternative embodiments, provided are methods for:

(i) predicting the successful construction of a genomic T-Cell Receptor (TCR) library from a tissue sample;

(ii) predicting the successful sequencing of a T-Cell Receptor (TCR) sequence from a tissue sample, or

(iii) predicting the success of assays used for quantifying T-cells and sequencing T-cell receptors, and the success of assays used to assess TCR quantity and clonality,

wherein optionally the tissue sample comprises or is derived from a biopsy or a Formalin-Fixation and Paraffin Embedding (FFPE) sample, and optionally the tissue sample is a human or a non-human (an animal) sample, and optionally the T cell is a tumor infiltrating lymphocyte (TIL),

comprising:

(a) providing an amplification primer pair, optionally a PCR primer pair, capable of amplifying a segment of DNA consisting of about 200 to about 400 base pairs (bp), or about 150 to about 350 bp, or about 100 to about 300 bp, or about 50 to about 200 bp, located on a genome from 0 to about 20,000 kilobases (kb), or from between about 10 to 10,000 bp, or about 20 to 5,000 bp, or about 50 to about 3,000 bp, 5′ upstream or 3′downstream of a TCR gene sequence, optionally a TCR alpha, beta, gamma or delta gene sequence,

(b) providing a tissue sample, wherein optionally the tissue sample comprises or is derived from a biopsy sample or a Formalin-Fixation and Paraffin Embedding (FFPE) sample, and optionally the tissue sample is a human sample or a non-human sample (e.g., an animal, optionally a domestic, a laboratory (e.g., mouse, rat, rabbit) or a commercial (e.g., a farm) animal), and optionally the sample comprises T cells, tumor infiltrating lymphocytes (TILs) or clonally expanded TILs or a combination thereof,

and optionally the biopsy comprises a needle or punch biopsy,

and optionally the biopsy sample comprises a tissue sample, a tissue comprising a dysfunctional cell, a tissue biopsy, a cancer tissue or tumor biopsy, wherein optionally the cancer or tumor is a colorectal cancer, ovarian cancer or glioblastoma biopsy,

and optionally the method comprises isolating or purifying, partially isolating or purifying, or substantially isolating or purifying, a DNA, or a genomic DNA, or a recombinant version thereof, comprising the segment or subseqence of DNA to be amplified;

(c) amplifying the segment or subseqence of DNA, or the genomic DNA, which optionally is in or is from or is derived from or is isolated or purified from, the tissue using the amplification primer pair, wherein optionally the amplification comprises use of polymerase chain reaction (PCR), and optionally the PCR comprises a real-time PCR (also known as quantitative polymerase chain reaction (qPCR)); and

(d) determining if the amplification primer pair completely, substantially completely, or partially amplified the segment of DNA, or the genomic DNA, wherein optionally the amplification primer pair is determined to (successfully) amplify the segment of DNA, or the genomic DNA, if a newly amplified DNA segment is detected or if a plurality of newly amplified DNA segments are detected (wherein optionally the newly amplified DNA was amplified from the completely, substantially completely, or partially amplified DNA),

wherein successful amplification of the segment of DNA, or the genomic DNA, by the amplification primer pair:

(i) predicts a successful construction of a genomic TCR library from the tissue sample, or predicts that there is a substantial likelihood that a genomic TCR library can be constructed from the tissue sample;

(ii) predicts the successful sequencing of a T-Cell Receptor (TCR) sequence from the tissue sample, or predicts that there is a substantial likelihood that a T-Cell Receptor (TCR) sequence can be sequenced, or accurately sequenced, from the genomic sample, or

(iii) predicts the success of assays used for quantifying T-cells and sequencing T-cell receptors, and the success of assays used to assess TCR quantity and clonality.

In alternative embodiments, by predicting the successful sequencing of a T-Cell Receptor (TCR) sequence from the tissue sample, or predicting that there is a substantial likelihood (wherein optionally the substantial likelihood is that there is a greater than 85%, 90%, 95%, 98% or 99% likelihood) that a T-Cell Receptor (TCR) sequence can be completely or partially sequenced, or accurately completely or partially sequenced, from the segment of DNA, or the genomic DNA, the method predicts the chance of success that a sequencing library can be constructed from the tissue sample or from a genomic sample derived from the tissue sample, or can predict the chance of success of sequencing of T-Cell receptor (TCR) DNA (genomic) sequences derived from the tissue sample, wherein optionally the sequencing of T-Cell receptor (TCR) DNA (genomic) sequences comprises use of an ImmunoSEQ™ Assay (Adaptive Biotechnologies, Seattle, Wash.).

In alternative embodiments, the PCR amplification primer pair comprises SEQ ID NO:1 and SEQ ID NO:2.

In alternative embodiments, the methods further comprise use of a positive control, wherein the positive control comprises use of a synthesized DNA nucleotide sequence (as internal control) representing the genomic segment (the amplicon) generated (amplified) by the amplification primer pair, where optionally the amplification primers in the positive control are run (used) at concentrations ranging from 10⁻⁶ to 10⁻¹⁸M, or from 10⁻⁷ to 10⁻¹⁷ M, or from 10⁻⁵ to 10⁻¹⁵ M.

In alternative embodiments, provided are kits or products of manufacture comprising materials used to practice a method as provided herein.

In alternative embodiments, provided are multiplexed systems, or high-throughput systems, comprising elements or materials used or needed to practice the method as provided herein, and optionally further comprising elements or materials for T-Cell Receptor (TCR) sequence library construction, e.g., including the PCR amplification primer pair comprises SEQ ID NO:1 and SEQ ID NO:2, and optionally further comprising elements or materials for sequencing of a TCR chain.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-B graphically illustrate two exemplary receiver operator characteristic (ROC) curves, which were employed to assess the performance of a ImmunoQC™ method as provided herein, where the ImmunoQC™ ROC for Formalin-Fixation and Paraffin Embedding (FFPE)-derived DNA is displayed as sensitivity % as a function of Specificity %, as described in further detail in Example 1, below.

Like reference symbols in the various drawings indicate like elements.

### DETAILED DESCRIPTION

In alternative embodiments, provided are compositions and methods for predicting the successful construction of genomic TCR libraries from tissue samples, and for predicting the successful sequencing of a T-Cell Receptor (TCR) sequence from that sample. Provided herein for the first time are cost-effective methods for accurately predicting the analytic success of T-cell quantification and T-cell receptor sequencing, e.g., prior to the use of an expensive assay such as the ImmunoSEQ™ Assay (Adaptive Biotechnologies, Seattle, Wash.). Screening tests as provided herein can save investigators, time, money, and precious reagents, including DNA extracted from limited resources, e.g., DNA samples from formalin-fixed, paraffin-embedded tumor samples, or any other source of degraded DNA. Provided herein for the first time are methods that employ nucleic acid amplification, e.g., real-time PCR, to predict the analytic success of methods for quantifying T-cell and sequencing T-cell receptors using a combination of primers, including using primers as provided herein.

In alternative embodiments, provided herein is a tractable, cost efficient method (so-called the “ImmunoQC™” assay) to predict successful library construction and sequencing (e.g., next-generation sequencing) of a TCR chain, optionally a genomic TCR chain sequence, including to predict successful library construction and sequencing of a TCR beta, alpha, gamma and/or delta chain sequence. The results of methods provided herein generate a prediction that can be utilized to determine whether commercially available assays (e.g., ImmunoSeq, Adaptive Biotechnologies, Seattle, Wash.) can effectively assess TCR quantity and clonality using T-cell genomic DNA samples, e.g., from biopsy or Formalin-Fixation and Paraffin Embedding (FFPE) samples, and thus will succeed, or not.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Compositions and Methods for Predicting the Successful Construction of Genomic TCR Libraries from Tissue Samples, and for Predicting the Successful Sequencing of a T-Cell Receptor (TCR) Sequence from that Sample

This example demonstrates that methods and compositions as provided herein, can effectively generate a prediction that can be utilized to determine whether commercially available assays (e.g., ImmunoSeq, Adaptive Biotechnologies, Seattle,

WA) can effectively assess TCR quantity and clonality using T-cell genomic DNA samples, e.g., from biopsy or Formalin-Fixation and Paraffin Embedding (FFPE) samples, and thus will succeed, or not. This example presents validating data that demonstrates that compositions and methods as provided herein are effective for predicting the successful construction of genomic TCR libraries from tissue samples, and for predicting the successful sequencing of a T-Cell Receptor (TCR) sequence from that sample.

Methods and compositions (e.g., kits) as provided herein (so-called “ImmunoQC™” assays) are real time genomic amplification (e.g., polymerase chain reaction-, or PCR-, based) methods that can predict the success of a genomic library, e.g., a next generation library construction from a DNA sample, e.g., from any tissue sample, e.g., from a biopsy or a Formalin-Fixation and Paraffin Embedding (FFPE) sample, and the methods and compositions as provided herein can predict the success of sequencing of a TCR sequence, optionally a TCR genomic sequence; thus, methods and compositions (e.g., kits) as provided herein (so-called “ImmunoQC™” assays) can assess or predict the success of a commercially available ImmunoSeg™ (Adaptive Biotechnologies, Seattle, Wash.) TIL assay.

### Primer Design and Real Time PCR Assay

The ImmunoSeg™ TIL assay involves the next-generation sequencing of a DNA fragment at the TCR beta locus in a genomic DNA tissue sample, e.g., from a biopsy sample. To assess the integrity of the genomic DNA in this region, and to thereby assess or predict the successful construction of genomic TCR libraries from these tissue samples, and for predicting the successful sequencing of a T-Cell Receptor (TCR) sequence from that sample, the following exemplary methods and proof of concept (validating) data comprise:

In one embodiment, PCR primers were designed to amplify a 146 base pair amplicon in the region bordering a TCR beta locus

Forward:
(SEQ ID NO: 1)
AGGCCTCAGTTTCCTTGCTC;
Reverse:
(SEQ ID NO: 2)
TGCTGTCTGTTGTTTCACCCT.

The region amplified by SEQ ID NO:1 and SEQ ID NO:2 is located at chromosome 7, positions 154,095,063 to 154,095,208 and has the following nucleotide sequence:

(SEQ ID NO: 3)
AGGCCTCAGTTTCCTTGCTCacagatgtggatcccaggagctgcctca
aatacttggaggcatgaatggttctaaatcacgcatgccatgcctgac
acattaaccccccattctcttcactgtgcAGGGTGAAACAACAGACAG
CA

In alternative embodiments, primers are designed to amplify about 100 to about 300 base pair (bp) regions (or about 50 to about 500 base pair (bp) regions) from 0 to about 20,000 kilobases (kb) 5′ upstream and/or 3′downstream of the TCR gene sequence (e.g., a TCR alpha, beta, gamma or delta gene sequence). These regions were selected to reflect the genomic integrity of the chromosomal region encompassing the TCR sequence and to avoid the variable regions of the TCR sequence which might confound effective genomic DNA amplification, e.g., PCR amplification.

Thus, in alternative embodiments, primers and amplified (e.g., PCR amplified) regions are:

T cell receptor alpha (and gamma)
(chr14:21,601,720-22,589,237)
>chr14:23589541 + 23589813 273 bp
(SEQ ID NO: 4)
TGGGCAAATGGGGTATACATTTCCCCTTTACCAGTGCATCTGGGCAAA
TGGGGTATACATctcctttttccccgtctggggagagaacaatagctt
tgtgagattcatggcccccaaagtccagcatatccggtacccctcctc
cctccttagcccatttgcttcctatgatggctgcaaagagagaaactt
gctgttggaaacagggggccttgggagtcaggctaagggattggacct
tcctcctcacccaatgaaactgagagaagaacctgcattttccctggg
gtggGATGCACTGGTAAAGGGGAA
T cell receptor beta
(chr7:142,268,284-142,871,093)
>chr7:154095063 - 154095208 146 bp
(SEQ ID NO: 5)
AGGCCTCAGTTTCCTTGCTCGCTGTCTGTTGTTTCACCCTAGGCCTCA
GTTTCCTTGCTCacagatgtggatcccaggagctgcctcaaatacttg
gaggcatgaatggttctaaatcacgcatgccatgcctgacacattaac
cccccattctcttcactgtgcAGGGTGAAACAACAGACAGCA
T cell receptor delta
(chr7:38,011,608-38,656,862)
>chr7:37656346 + 37656475 130 bp
(SEQ ID NO: 6)
GGCATCAGCCTATTGCATTTCTGTTGCCTGCAATTCCTTTGGCATCAG
CCTATTGCATTTacttatttgcctaaaatgtgatccttggttagaagc
catgtatcattatgatgaataaggattttgttaagtccatggatggta
ttgctAAAGGAATTGCAGGCAACAG

With real time PCR, both DNA integrity and quantity can be simultaneously assessed, as reflected in the threshold cycle (Ct) of the real time PCR assay.

Below we document the performances of the ImmunoQC™ method employing the primer set for the TCR beta chain locus to predict success or failure of next generation sequencing of the TCR beta locus as achieved with the commercially available ImmunoSeg™ assay (Adaptive Biotechnologies, Seattle, Wash.).

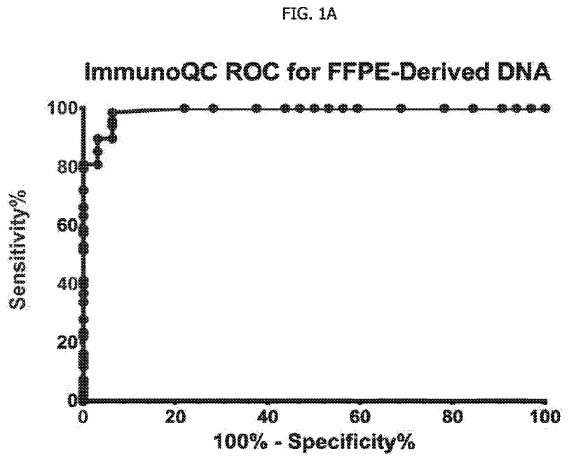

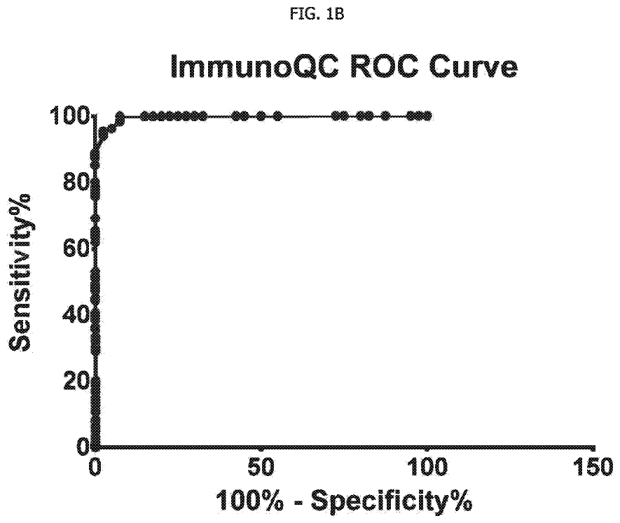

For the data listed in Table 1a (see also FIG. 1A), a set of 100 FFPE-derived DNA samples which had either previously failed (N=32) or succeeded (N=68) in the Adaptive ImmunoSeq TIL assay was used.

For the data listed in Table 1b (see also FIG. 1B), a set of 177 FFPE-derived DNA samples which had either previously failed (N=40) or succeeded (N=137) in the Adaptive ImmunoSeq TIL assay was used.

In these same real time PCR assays a synthesized DNA nucleotide sequence (internal control) representing the amplicon generated by the above primers were run at range of concentrations (10⁻⁷ to 10⁻¹⁷ M).

For the data listed in Table 1a (see also FIG. 1A), a concentration of the internal control was then identified (10⁻¹⁶ M) below which all DNA samples failed or above which all succeeded in the ImmunoSeg™ (Adaptive Biotechnologies, Seattle, Wash.) TIL Assay. For the exemplary ImmunQC™ assay (an exemplary assay as provided herein), a pass/fail value, deltaCt, was then defined as [Ct(internal control)-Ct(sample)]. For the data listed in Table 1a, for any sample, if the deltaCt>0.25, a pass value was assigned, and if the deltaCt≤0.25, a fail value was assigned.

For the data listed in Table 1b (see also FIG. 1B), a concentration of the internal control was then identified (10⁻¹⁵ M) below which all DNA samples failed or above which all succeeded in the ImmunoSeg™ (Adaptive Biotechnologies, Seattle, Wash.) TIL Assay. For the ImmunQC™ assay, a pass/fail value, deltaCt, was then defined as [Ct(internal control)-Ct(sample)]. For any sample, if the deltaCt≥0.35, a pass value was assigned, and if the deltaCt<0.35, a fail value was assigned.

### Performance of the ImmunoQC™ Method to Predict Success of the ImmunoSeg™ (Adaptive Biotechnologies, Seattle, Wash.) Assay

The receiver operator characteristic (ROC) curve was employed to assess the performance of the ImmunoQC™ method. A range of sensitivity and specificity values were calculated for the 100 sample set containing samples which either failed or succeeded in the ImmunoSeg™ assay and the ImmunoQC™ assay (see appendix, below, for calculations). In one exemplary test, the area under the curve was calculated to be 0.9897 with a standard error of 0.007504 (P<0.0001) indicating near perfect performance of the ImmunoQC™ method, as listed and graphically illustrated in Table 1a (below) and FIG. 1a, respectively. In another exemplary test, the area under the curve was calculated to be 0.9963 with a standard error of 0.002755 (P<0.0001) indicating near perfect performance of the ImmunoQC™ method, as illustrated in Table 1b (below) and as graphically illustrates in FIG. 1b, respectively.

### Cost and Tractability Analysis

The reagent cost associated with assays as provided herein (the so-called ImmunoQC™ assay) is modest, e.g., approximately $0.70, per sample, compared with the overall cost of constructing a next-generation sequencing library and to sequence the library.

### High Throughput Systems

In alternative embodiments, assays as provided herein (the so-called ImmunoQC™ assay) can be adapted to any high throughput, robotic platform, e.g., a platform compatible with the already existing sample configuration of the ImmunoSeg™ Assay (96 well plate) as well as other existing platforms, including platforms that are commonly used with high throughput next-generation sequencing platforms.

In alternative embodiments, assays as provided herein are designed and configured as multiplexed systems, e.g., including high throughput, robotic platforms.

### Materials and Methods

### Real Time PCR Protocol:

### PCR Primers:

Forward:
(SEQ ID NO: 1)
AGGCCTCAGTTTCCTTGCTC;
Reverse:
(SEQ ID NO: 2)
TGCTGTCTGTTGTTTCACCCT.

Primers were combined into a Primer Mix at final concentration 2.5 uM.

### PCR Master Mix:

### Power SYBR Green PCR Master Mix

### Applied Biosystems: Catalog #4367659

### Real Time PCR Reaction:

| | |
|---|---|
| DNA | 2.0 ul |
| Primer Mix | 1.5 ul |
| Master Mix | 10.0 ul |
| H₂O | 6.5 ul |
| Total Volume | 20.0 ul |

### Real Time PCR Conditions

Initial Denaturation: 10 minutes @ 95° C.
Amplification (40 cycles):

15 seconds @ 95° C.

60 seconds @ 60° C.

TABLE 1a
Sensitivity and Specificity Calculations

| Cutoff | | |
|---|---|---|
| Delta Ct = [Ct (internal | | |
| control)-Ct (sample)] | Sensitivity % | Specificity % |
| >−5.750 | 100 | 3.125 |
| >−4.250 | 100 | 6.25 |
| >−3.750 | 100 | 9.375 |
| >−3.250 | 100 | 15.63 |
| >−2.750 | 100 | 21.88 |
| >−2.250 | 100 | 31.25 |
| >−1.950 | 100 | 40.63 |
| >−1.800 | 100 | 43.75 |
| >−1.600 | 100 | 46.88 |
| >−1.400 | 100 | 50 |
| >−1.200 | 100 | 53.13 |
| >−1.050 | 100 | 56.25 |
| >−0.7500 | 100 | 62.5 |
| >−0.2500 | 100 | 71.88 |
| >0.1000 | 100 | 78.13 |
| >0.2500 | 98.53 | 93.75 |
| >0.3500 | 95.59 | 93.75 |
| >0.4500 | 94.12 | 93.75 |
| >0.6000 | 89.71 | 93.75 |
| >0.8500 | 89.71 | 96.88 |
| >1.100 | 85.29 | 96.88 |
| >1.250 | 80.88 | 96.88 |
| >1.350 | 80.88 | 100 |
| >1.450 | 79.41 | 100 |
| >1.600 | 72.06 | 100 |
| >1.750 | 66.18 | 100 |
| >1.900 | 63.24 | 100 |
| >2.100 | 58.82 | 100 |
| >2.250 | 57.35 | 100 |
| >2.350 | 52.94 | 100 |
| >2.450 | 51.47 | 100 |
| >2.650 | 41.18 | 100 |
| >2.900 | 39.71 | 100 |
| >3.050 | 36.76 | 100 |
| >3.150 | 33.82 | 100 |
| >3.250 | 27.94 | 100 |
| >3.350 | 23.53 | 100 |
| >3.450 | 22.06 | 100 |
| >3.550 | 16.18 | 100 |
| >3.650 | 14.71 | 100 |
| >3.750 | 13.24 | 100 |
| >3.900 | 11.76 | 100 |
| >4.400 | 7.353 | 100 |
| >4.900 | 5.882 | 100 |
| >5.250 | 4.412 | 100 |
| >6.000 | 2.941 | 100 |
| >7.000 | 1.471 | 100 |

TABLE 1b
Sensitivity and Specificity Calculations

| Cutoff Delta Ct [Ct | | |
|---|---|---|
| (internal Contol)-Ct | | |
| (sample)] | Sensitivity % | Specificity % |
| >−5.305 | 100 | 2.5 |
| >−4.650 | 100 | 5 |
| >−3.750 | 100 | 12.5 |
| >−3.000 | 100 | 17.5 |
| >−2.400 | 100 | 20 |
| >−2.200 | 100 | 25 |
| >−2.050 | 100 | 27.5 |
| >−1.950 | 100 | 45 |
| >−1.700 | 100 | 50 |
| >−1.400 | 100 | 55 |
| >−1.150 | 100 | 57.5 |
| >−0.7750 | 100 | 67.5 |
| >−0.5250 | 100 | 70 |
| >−0.4300 | 100 | 72.5 |
| >−0.3000 | 100 | 75 |
| >−0.1700 | 100 | 77.5 |
| >−0.0500 | 100 | 80 |
| >0.0500 | 100 | 82.5 |
| >0.1500 | 100 | 85 |
| >0.3500 | 100 | 92.5 |
| >0.6500 | 99.27 | 92.5 |
| >0.9000 | 98.54 | 92.5 |
| >1.100 | 96.35 | 95 |
| >1.250 | 95.62 | 97.5 |
| >1.350 | 94.89 | 97.5 |
| >1.450 | 94.16 | 97.5 |
| >1.570 | 89.05 | 100 |
| >1.670 | 88.32 | 100 |
| >1.750 | 87.59 | 100 |
| >1.900 | 85.4 | 100 |
| >2.050 | 80.29 | 100 |
| >2.200 | 78.83 | 100 |
| >2.320 | 78.1 | 100 |
| >2.370 | 77.37 | 100 |
| >2.435 | 76.64 | 100 |
| >2.485 | 75.91 | 100 |
| >2.650 | 69.34 | 100 |
| >2.810 | 65.69 | 100 |
| >2.850 | 64.96 | 100 |
| >2.890 | 64.23 | 100 |
| >2.915 | 63.5 | 100 |
| >2.965 | 62.04 | 100 |
| >3.050 | 53.28 | 100 |
| >3.120 | 51.82 | 100 |
| >3.170 | 51.09 | 100 |
| >3.210 | 49.64 | 100 |
| >3.260 | 48.91 | 100 |
| >3.335 | 48.18 | 100 |
| >3.385 | 47.45 | 100 |
| >3.410 | 45.26 | 100 |
| >3.460 | 44.53 | 100 |
| >3.505 | 40.88 | 100 |
| >3.525 | 40.15 | 100 |
| >3.565 | 39.42 | 100 |
| >3.595 | 38.69 | 100 |
| >3.640 | 36.5 | 100 |
| >3.690 | 35.77 | 100 |
| >3.725 | 33.58 | 100 |
| >3.760 | 32.85 | 100 |
| >3.785 | 32.12 | 100 |
| >3.830 | 30.66 | 100 |
| >3.880 | 29.93 | 100 |
| >3.950 | 29.2 | 100 |
| >4.050 | 20.44 | 100 |
| >4.150 | 19.71 | 100 |
| >4.260 | 18.98 | 100 |
| >4.380 | 17.52 | 100 |
| >4.470 | 16.79 | 100 |
| >4.525 | 15.33 | 100 |
| >4.605 | 14.6 | 100 |
| >4.665 | 13.87 | 100 |
| >4.685 | 13.14 | 100 |
| >4.710 | 11.68 | 100 |
| >4.760 | 10.95 | 100 |
| >4.875 | 8.759 | 100 |
| >4.975 | 8.029 | 100 |
| >5.250 | 6.569 | 100 |
| >5.620 | 5.839 | 100 |
| >5.770 | 5.109 | 100 |
| >5.900 | 3.65 | 100 |
| >6.100 | 2.92 | 100 |
| >6.300 | 2.19 | 100 |
| >6.850 | 1.46 | 100 |
| >7.650 | 0.7299 | 100 |

A number of embodiments of the invention have been described.

Nevertheless, it can be understood that various modifications may be made without departing from the spirit and scope of the invention. Accordingly, other embodiments are within the scope of the following claims.

1. A method for:
(i) predicting the successful construction of a genomic T-Cell Receptor (TCR) library from a tissue sample;
(ii) predicting the successful sequencing of a T-Cell Receptor (TCR) sequence from a tissue sample, or
(iii) predicting the success of assays used for quantifying T-cells and sequencing T-cell receptors, and the success of assays used to assess TCR quantity and clonality,
comprising:
(a) providing an amplification primer pair capable of amplifying a segment of DNA consisting of about 200 to about 400 base pairs (bp), located on a genome from 0 to about 20,000 kilobases (kb), 5′ upstream or 3′downstream of a TCR gene sequence,
(b) providing a tissue sample;
(c) amplifying the segment of DNA, or the genomic DNA; and
(d) determining if the amplification primer pair amplified the segment of DNA, or the genomic DNA,
wherein successful amplification of the segment of DNA, or the genomic DNA, by the amplification primer pair:
(i) predicts a successful construction of a genomic TCR library from the tissue sample, or predicts that there is a substantial likelihood that a genomic TCR library can be constructed from the tissue sample;
(ii) predicts the successful sequencing of a T-Cell Receptor (TCR) sequence from the tissue sample, or predicts that there is a substantial likelihood that a T-Cell Receptor (TCR) sequence can be sequenced, or accurately sequenced, from the genomic sample, or
(iii) predicts the success of assays used for quantifying T-cells and sequencing T-cell receptors, and the success of assays used to assess TCR quantity and clonality.

2. The method of claim 1, wherein by predicting the successful sequencing of a T-Cell Receptor (TCR) sequence from the tissue sample, or predicting that there is a substantial likelihood that a T-Cell Receptor (TCR) sequence can be sequenced, or accurately sequenced, from the segment of DNA, or the genomic DNA, the method predicts the success that a sequencing library can be constructed from the tissue sample or from a genomic sample derived from the tissue sample, or can predict the success of sequencing of T-Cell receptor (TCR) DNA (genomic) sequences derived from the tissue sample, wherein optionally the sequencing of T-Cell receptor (TCR) DNA (genomic) sequences comprises use of an ImmunoSEQ™ Assay (Adaptive Biotechnologies, Seattle, Wash.).

3. The method of claim 1, wherein the PCR amplification primer pair comprises SEQ ID NO:1 and SEQ ID NO:2.

4. The method of claim 1, further comprising use of a positive control comprising use of a synthesized DNA nucleotide sequence (as internal control) representing the genomic segment (the amplicon) generated (amplified) by the amplification primer pair, where optionally the amplification primers in the positive control are run (used) at concentrations ranging from 10⁻⁷ to 10⁻¹⁷ M.

5. A kit or product of manufacture comprising materials used to practice the method of claim 1.

6. A multiplexed system, or a high-throughput system, comprising elements or materials to practice the method of claim 1, and optionally further comprising elements or materials for T-Cell Receptor (TCR) sequence library construction, and optionally further comprising elements or materials for sequencing of a TCR chain.

7. The method of claim 1, wherein the tissue sample comprises or is derived from a biopsy or a Formalin-Fixation and Paraffin Embedding (FFPE) sample, and optionally the tissue sample is a human or a non-human, optionally an animal, sample, and optionally the T cell is a tumor infiltrating lymphocyte (TIL).

8. The method of claim 1, wherein the amplification primer pair capable of amplifying a segment of DNA consists of about 150 to about 350 bp, or about 100 to about 300 bp, or about 50 to about 200 bp.

9. The method of claim 1, wherein the amplification primer pair is capable of amplifying a segment of DNA is located on a genome from between about 10 to 10,000 bp, or about 20 to 5,000 bp, or about 50 to about 3,000 bp, 5′ upstream or 3′downstream of a TCR gene sequence.

10. The method of claim 1, wherein the amplification primer pair comprises a polymerase chain reaction (PCR) primer pair.

11. The method of claim 1, wherein the TCR or TCR gene sequence comprises a TCR alpha, beta, gamma or delta gene sequence.

12. The method of claim 1, wherein the tissue sample comprises or is derived from a biopsy sample or a Formalin-Fixation and Paraffin Embedding (FFPE) sample, or equivalents.

13. The method of claim 1, wherein the tissue sample is a human sample.

14. The method of claim 1, wherein the sample comprises T cells, tumor infiltrating lymphocytes (TILs), clonally expanded TILs, or any combination thereof.

15. The method of claim 1, wherein the biopsy comprises a needle or punch biopsy.

16. The method of claim 1, wherein the biopsy sample comprises a tissue sample, a cancer tissue biopsy, or a colorectal cancer, ovarian cancer or glioblastoma biopsy.

17. The method of claim 1, wherein the method comprises isolating or purifying, partially isolating or purifying, or substantially isolating or purifying, a DNA, or a genomic DNA, or a recombinant version thereof, comprising the segment of DNA to be amplified.

18. The method of claim 1, wherein the amplified segment of DNA, or the genomic DNA is in or is from or is derived from or is isolated from, the tissue using the amplification primer pair, wherein optionally the amplification comprises use of PCR, optionally real-time PCR, or also known as quantitative polymerase chain reaction (qPCR).

19. The method of claim 1, wherein the amplification primer pair is determined to successfully amplify the segment of DNA, or the genomic DNA, if a newly amplified DNA segment is detected or if a plurality of newly amplified DNA segments are detected.
